(19) United States
(12) Patent Application Publication
(10) Pub. No.: US 2024/0013901 A1
(43) Pub. Date: Jan. 11, 2024

(54) SYSTEMS AND METHODS FOR PLANNING A MEDICAL ENVIRONMENT

(71) Applicant: INTUITIVE SURGICAL OPERATIONS, INC., Sunnyvale, CA (US)

(72) Inventors: Prasad V. Upadrasta, San Jose, CA (US); John Ryan Steger, Los Gatos, CA (US)

(21) Appl. No.: 18/255,314

(22) PCT Filed: Nov. 29, 2021
(86) PCT No.: PCT/US2021/060963

§ 371 Date: May 31, 2023

Related U.S. Application Data
(60) Provisional application No. 63/120,140, filed on Dec. 1, 2020.

(51) Int. Cl. G16H 40/40

(52) U.S. Cl. CPC G16H 40/40

(57) ABSTRACT

A system may comprise a processor and a memory having computer readable instructions stored thereon. The computer readable instructions, when executed by the processor, may cause the system to receive spatial information for of a medical environment; determine a component for use in the medical environment; receive an indicator for a mode of operation of the component; receive a set of operation constraints for the component for the mode of operation; and generate an environment preparation plan based on the set of operation constraints and the spatial information.

### CROSS-REFERENCED APPLICATIONS

This application claims the benefit of U.S. Provisional Application 63/120,140 filed Dec. 1, 2020, which is incorporated by reference herein in its entirety.

This application incorporates by reference in their entireties U.S. Provisional Application No. 63/120,175, filed Dec. 1, 2020, titled “SYSTEMS AND METHODS FOR GENERATING VIRTUAL REALITY GUIDANCE” and U.S. Provisional Application No. 63/120,191, filed Dec. 1, 2020, titled “SYSTEMS AND METHODS FOR GENERATING AND EVALUATING A MEDICAL PROCEDURE.”

### FIELD

The present disclosure is directed to systems and methods for robot-assisted medical procedures and more specifically to developing a medical environment plan based on a mode of operation for a robot-assisted medical system.

### BACKGROUND

Planning tools for the set-up, operation, trouble-shooting, maintenance, and storage of teleoperational robotic or robot-assisted systems are often generic and may be unable to anticipate the unique circumstances of a particular medical environment, including the dimensions of the operating space, the robot-assisted system equipment available in the environment, the auxiliary equipment available in the environment, the location of utilities in the environment, the personnel in the environment, and other parameters associated with the robot-assisted system. Systems and methods are needed to assist medical personnel by providing mode-specific medical environment plans that are customized to the components and constraints of a particular medical environment.

### SUMMARY

The embodiments of the invention are best summarized by the claims that follow the description.

Consistent with some embodiments, a system may comprise a processor and a memory having computer readable instructions stored thereon. The computer readable instructions, when executed by the processor, may cause the system to receive spatial information for of a medical environment; determine a component for use in the medical environment; receive an indicator for a mode of operation of the component; receive a set of operation constraints for the component for the mode of operation; and generate an environment preparation plan based on the set of operation constraints and the spatial information.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a flowchart illustrating a method for generating virtual guidance according to some embodiments.

FIGS. 2A and 2B illustrates spatial information for a medical environment according to some embodiments.

FIGS. 3A and 3B illustrates a menu of components for use in the medical environment.

FIG. 4 is a schematic illustration of a robot-assisted medical system according to some embodiments.

FIG. 5 illustrates a menu of component modes of operation.

FIG. 6 illustrates an environment preparation plan for a medical environment according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

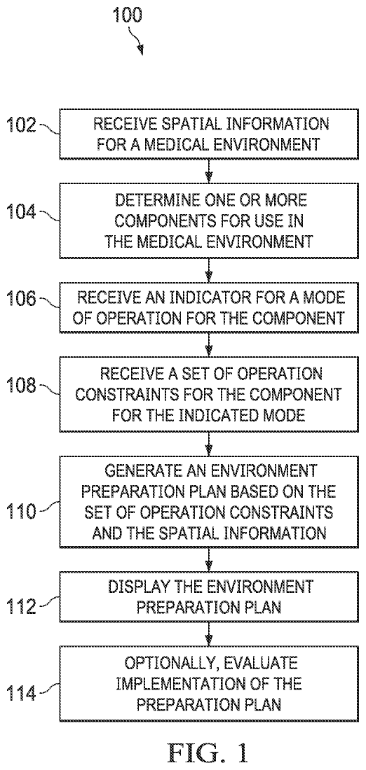

Planning tools may assist in the efficient, safe, and effective use of robot-assisted systems in a medical environment. A well-planned medical environment may anticipate the range of motion, staff interactions, and utility access needs associated with medical components with various modes of use within a medical environment. As a result, the duration of medical environment use may become more predictable and may promote more efficient scheduling of space and resources. As described below, mode-based planning tools that incorporate physical parameters and constraints for components in the medical environment may be used to generate a more detailed and customized environment preparation plan. FIG. 1 is a flowchart illustrating a method 100 for generating an environment preparation plan according to some embodiments. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the processes may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes may be performed by a control system.

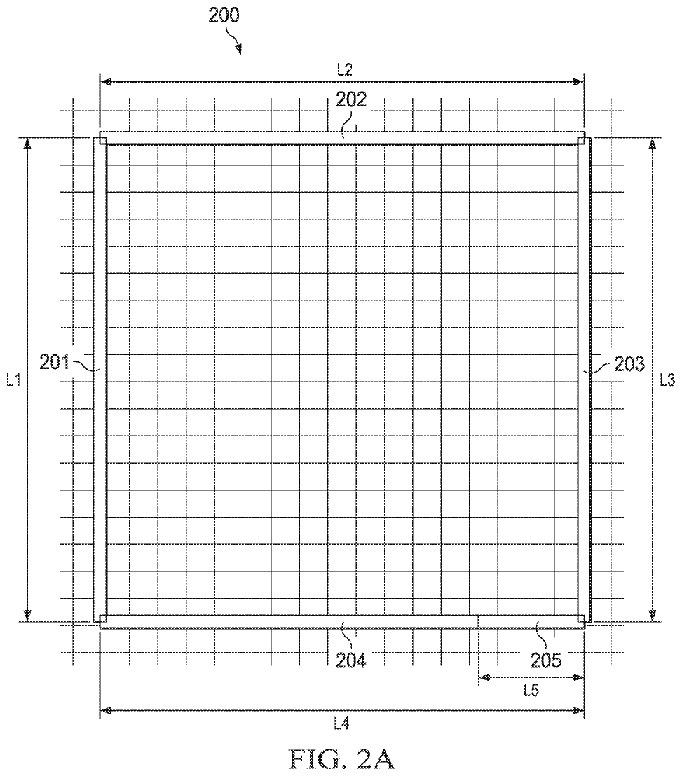

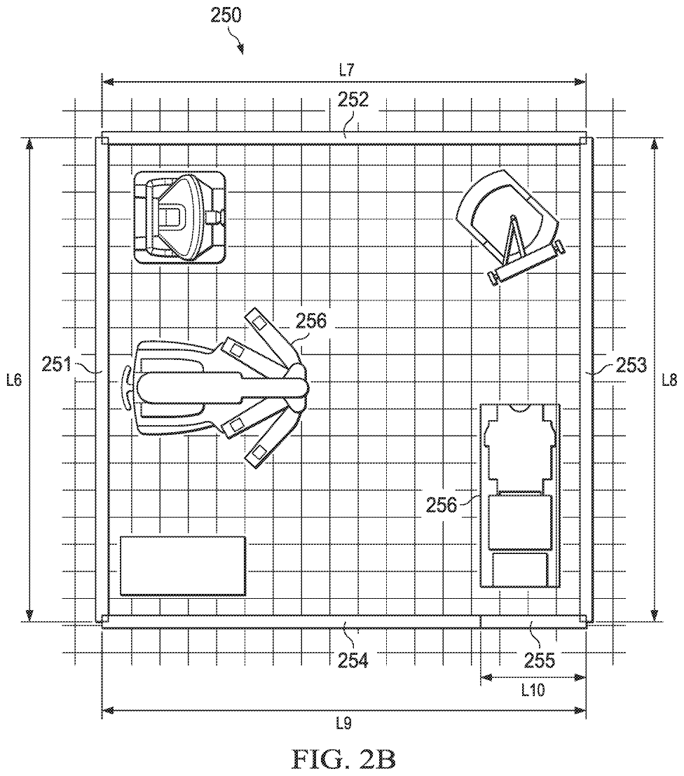

At a process 102, spatial information for a medical environment is received. The spatial information may include information about the two-dimensional or three-dimensional size, shape, and configuration of the medical environment. The spatial information may be received as linear dimensions (e.g., length, width, height), orientation information, coordinate frame positions, two-dimensional or three-dimensional image data, two-dimensional or three-dimensional model data, absolute or relative position/orientation information, or other types of information about the medical environment space. In some embodiments, the spatial information may be received from a mobile device such as a phone, tablet, camera, laptop or other portable measurement device that may measure, scan, image or otherwise record spatial information about the medical environment from within or proximate to the medical environment. The mobile device may include an application that prompts a user to record or capture spatial information. In one example, as illustrated in FIG. 2A, a medical environment 200 may have a length dimension L1 for a wall 201, a length dimension L2 for a wall 202, a length dimension L3 for a wall 203, a length dimension L4 for a wall 204, and a length dimension L5 for a door 205. The dimensions L1-L5 may be measured using a three-dimensional depth mapping system which may include a rangefinder, lidar system, camera, or other measurement tool that may a single purpose device or may be incorporated in to a mobile device such as a phone, tablet, or laptop. Other mapping systems, including sensor systems (e.g. electromagnetic position sensors, optical sensors) for tracking static or dynamic locations of components in the medical environment may be used. Any of the three-dimensional mapping technologies may be used alone or in combination with others. The medical environment 200 of FIG. 2A may be substantially empty of components or auxiliary equipment. In another example, as shown in FIG. 2B, a medical environment 250 includes medical components 256. The medical environment 250 may have a length dimension L6 for a wall 251, a length dimension L7 for a wall 252, a length dimension L8 for a wall 253, a length dimension L9 for a wall 254, and a length dimension L10 for a door 255. The dimensions L1-L5 may be measured using measurement tool as described above. Additionally, the measurement tool may record the presence, positions, and orientations of the medical components 256. In some embodiments, the spatial information may be received by an imaging or measurement system fixedly mounted in the medical environment. In some embodiments, the spatial information may be received from stored spatial information catalog about the medical environment such as a database of maps, models, or spatial information corresponding to predetermined rooms within a predetermined facility.

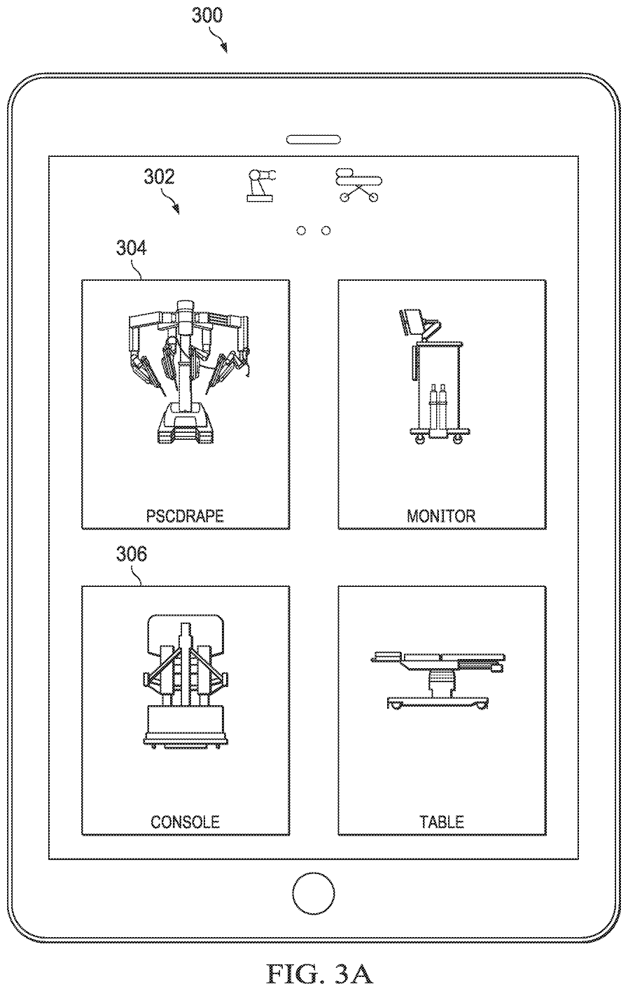

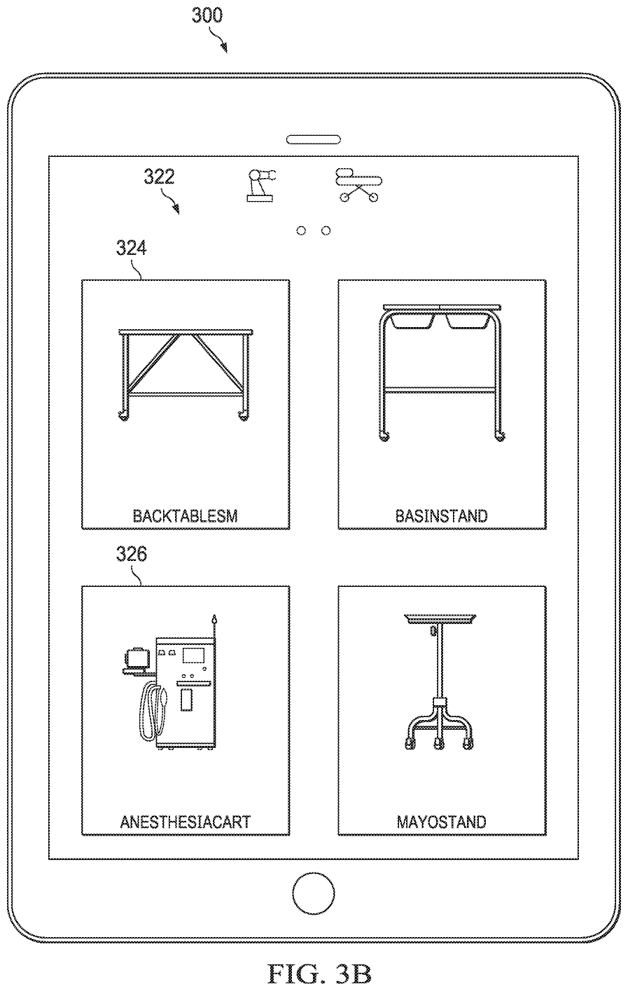

Referring again to FIG. 1, at a process 104, one or more components to be used in the medical environment may be determined. For example, the determination may be made based upon a user input received, for example, on a mobile device. The user may indicate one or more components to be located in the medical environment. As shown in FIG. 3A, a user display and input device 300, such as a touch screen display on a mobile phone, may provide a menu 302 of networked medical components such as a draped robot-assisted manipulator assembly 304 and an operator console 306 that a user may choose to deploy in the medical environment. As shown in FIG. 3B, the user input device 300 may provide another menu 322 of auxiliary components such as a table 324 and an anesthesia cart 326 that the user may choose to deploy in the medical environment in support of the networked medical components. In some embodiments, the medical environment may be empty and the components to be used may be moved into the medical environment. In some embodiments, one or more of the components may be present within the medical environment. An initial determination of the components in the medical environment may be made based upon analysis of the received spatial information. For example, image data from a camera or lidar data from a lidar scanner may be analyzed to recognize components in the medical environment when the spatial was gathered. A determination may also be made regarding whether the component determined to be in the medical environment is stationary or movable.

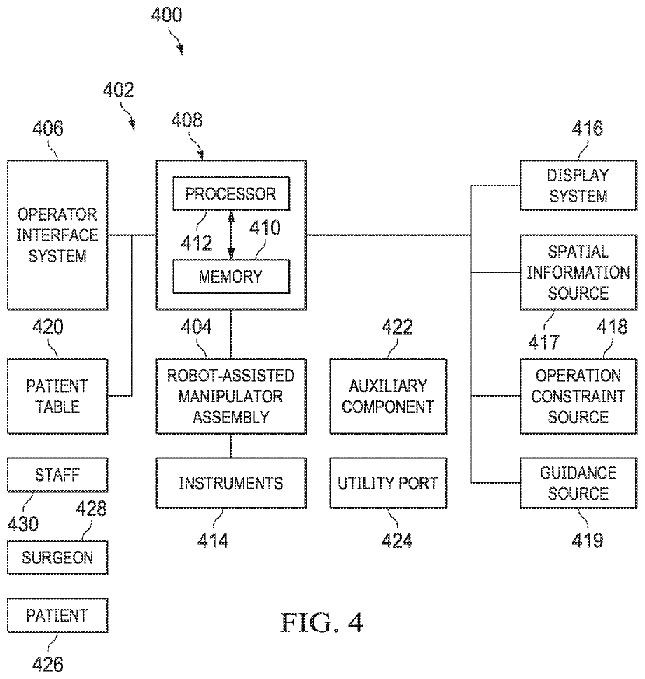

A medical environment may be used for multiple modes of operation of a robot-assisted medical system including for example, a preparation mode that may include a sterile preparation mode or procedure. The sterile preparation mode may include, for example, a draping mode or procedure. The multiple modes may also include a medical procedure or clinical mode, a trouble-shooting mode, a servicing mode, an inspection mode, a cleaning mode, and a storage mode. Any of a variety of components, systems, and people may occupy the medical environment depending on the mode of operation of the robot-assisted medical system. FIG. 4 illustrates a medical environment 400 having a medical environment frame of reference (X_{M}, Y_{M}, Z_{M}) including a robot-assisted medical system 402 that may include components such as a robot-assisted manipulator assembly 404, an operator interface system 406, and a control system 408. In one or more embodiments, the system 402 may be a robot-assisted medical system that is under the teleoperational control of a surgeon. In alternative embodiments, the medical system 402 may be under the partial control of a computer programmed to perform the medical procedure or sub-procedure. In still other alternative embodiments, the medical system 402 may be a fully automated medical system that is under the full control of a computer programmed to perform the medical procedure or sub-procedure with the medical system 402. One example of the medical system 402 that may be used to implement the systems and techniques described in this disclosure is the da Vinci® Surgical System manufactured by Intuitive Surgical Operations, Inc. of Sunnyvale, California. The medical environment 400 may be an operating room, a surgical suite, a medical procedure room, or other environment where medical procedures or medical training occurs.

The control system 408 may include at least one memory 410 and a processing unit including at least one processor 412 for effecting communication, control, and data transfer between components in the medical environment. Any of a wide variety of centralized or distributed data processing architectures may be employed in the control system 408. Similarly, the programmed instructions may be implemented as a number of separate programs or subroutines, or they may be integrated into a number of other aspects of the systems described herein, including teleoperational systems. In one embodiment, the control system 408 may support any of a variety of wired communication protocols or wireless communication protocols such as Bluetooth, IrDA, HomeRF, IEEE 802.11, DECT, and Wireless Telemetry. In some embodiments, the control system 408 may be in a different environment, partially or entirely remote from the manipulator assembly 404 and the operator interface system 406, including a different area of common surgical environment, a different room, or a different building.

The manipulator assembly 404 may be referred to as a patient side cart. One or more medical instruments 414 (also referred to as a tools) may be operably coupled to the manipulator assembly 404. The medical instruments 414 may include end effectors having a single working member such as a scalpel, a blunt blade, a needle, an imaging sensor, an optical fiber, an electrode, etc. Other end effectors may include multiple working members, and examples include forceps, graspers, scissors, clip appliers, staplers, bipolar electrocautery instruments, etc. The number of medical instrument 414 used at one time will generally depend on the medical procedure and the space constraints within the operating room among other factors. A medical instrument 414 may also include an imaging device. The imaging instrument may comprise an endoscopic imaging system using optical imaging technology, or comprise another type of imaging system using other technology (e.g. ultrasonic, fluoroscopic, etc.). The manipulator assembly 404 may include a kinematic structure of one or more links coupled by one or more non-servo controlled joints, and a servo-controlled robotic manipulator. In various implementations, the non-servo controlled joints can be manually positioned or locked, to allow or inhibit relative motion between the links physically coupled to the non-servo controlled joints. The manipulator assembly 404 may include a plurality of motors that drive inputs on the medical instruments 414. These motors may move in response to commands from the control system 408. The motors may include drive systems which when coupled to the medical instrument 414 may advance the medical instrument into a naturally or surgically created anatomical orifice in a patient. Other motorized drive systems may move the distal end of the medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the motors can be used to actuate an articulable end effector of the instrument for grasping tissue in the jaws of a biopsy device or the like. Kinematic information about the manipulator assembly 404 and/or the instruments 414 may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

The operator interface system 406 allows an operator such as a surgeon or other type of clinician to view images of or representing the procedure site and to control the operation of the medical instruments 414. In some embodiments, the operator interface system 406 may be located in the same room as a patient during a surgical procedure. However, in other embodiments, the operator interface system 406 may be located in a different room or a completely different building from the patient. The operator interface system 406 may generally include one or more control device(s) for controlling the medical instruments 414. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and the like. In some embodiments, the control device(s) will be provided with the same degrees of freedom as the medical tools of the robotic assembly to provide the operator with telepresence; that is, the operator is provided with the perception that the control device(s) are integral with the tools so that the operator has a sense of directly controlling tools as if present at the procedure site. In other embodiments, the control device(s) may have more or fewer degrees of freedom than the associated medical tools and still provide the operator with telepresence. In some embodiments, the control device(s) are manual input devices which move with six degrees of freedom, and which may also include an actuatable handle for actuating medical tools (for example, for closing grasping jaw end effectors, applying an electrical potential to an electrode, capture images, delivering a medicinal treatment, and the like). The manipulator assembly 404 may support and manipulate the medical instrument 414 while an operator views the procedure site through a display on the operator interface system 406. An image of the procedure site can be obtained by the imaging instrument, such as a monoscopic or stereoscopic endoscope, which can be manipulated by the manipulator assembly 404.

Another component that may, optionally, be arranged in the medical environment 400 is a display system 416 that may be communicatively coupled to the control system 408. The display system 416 may display, for example, images, instructions, and data for conducting a robot-assisted procedure. Information presented on the display system 416 may include endoscopic images from within a patient anatomy, guidance information, patient information, and procedure planning information. In some embodiments, the display system may be supported by an electronics cart that allows for mobile positioning of the display system.

A spatial information source 417 may be communicatively coupled to the control system 408. The spatial information source 417 may capture and/or store spatial information before it is sent to and received at process 102. In some embodiments, the spatial information source may be a mobile device such as a phone, tablet, camera, laptop or other portable measurement device that may measure, scan, image or otherwise record spatial information about the medical environment from within or proximate to the medical environment. The mobile device may include an application that prompts a user to record or capture spatial information. The spatial information source may include a camera, scanner, or other imaging device located in or capable of recording an image of the medical environment 400. In some embodiments, the spatial information source 417 may be a portable device supported by a surgeon 428 or staff member 430. Additionally or alternatively, the spatial information source 417 may be mounted to the walls, floor, ceiling, or other components in the medical environment 400. In some embodiments, the spatial information source may include a lidar scanning system that may scan the environment to generate three-dimensional images using reflected laser light. The spatial information source 417 may record and/or store two-dimensional or three-dimensional size, shape, and configuration data for the medical environment. The spatial information may be received as linear dimensions (e.g., length, width, height), orientation information, coordinate frame positions, image data, model data, and absolute or relative position/orientation information, or other types of information about the medical environment space.

An operation constraint source 418 may be communicatively coupled to the control system 408 or may be stored in the memory 410. The operation constraint source 418 may be a database stored outside of the medical environment 400 and accessed by the control system 408. The operation constraint source 418 may include stored information including operation constraints for each mode of operation of a component in the medical environment 400. For example, the operation constraints may include kinematic information for each component, information about the size and shape of the spatial envelope needed for the mode of operation, needed auxiliary equipment, needed utility access, needed personnel access, needed traffic flow access, patient information and requirements, and/or staffing requirements. Operation constraints may also include, for example, information about utility constraints including locations of outlets, such as AC outlets in the environment, lengths of connections such as electrical cords or cables, and distances between components that are connected by physical interconnects such as cables (e.g., fiber optic cables, power cables, data transfer cables, endoscope cables) and cords.

A guidance source 419 may be communicatively coupled to the control system 408 or may be stored in the memory 410. The guidance source 419 may include stored information including models, best practice information and historical procedure information for preparing a medical environment for a variety of operational modes. For example, the guidance source may include sample medical environment layouts for various procedures. Additionally or alternatively the guidance source 419 may include real-time personnel consultation including an advisory operator which may include experts, trainers, mentors, or other guidance staff that may support a user experience. The advisory operator may be remote and take the form of tele-connected trainers, mentors, supervisors, or other experts who may, for example, provide guidance in the form of instructions, suggestions, options, advisories, and warnings.

Other components in the medical environment 400 that may or may not be communicatively coupled to the control system 408 may include a patient table 420 and an auxiliary component 422 such as an instrument table, an instrument basin, an anesthesia cart, a supply cart, a cabinet, and seating. Other components in the medical environment 400 that may or may not be communicatively coupled to the control system 408 may include may include utility ports 424 such as electrical, water, and pressurized air outlets.

People in or capable of entering the medical environment 400 may include the patient 426 who may be positioned on the patient table 420, a surgeon 428 who may access the operator interface system 406, and staff members 430 which may include, for example surgical staff or maintenance staff.

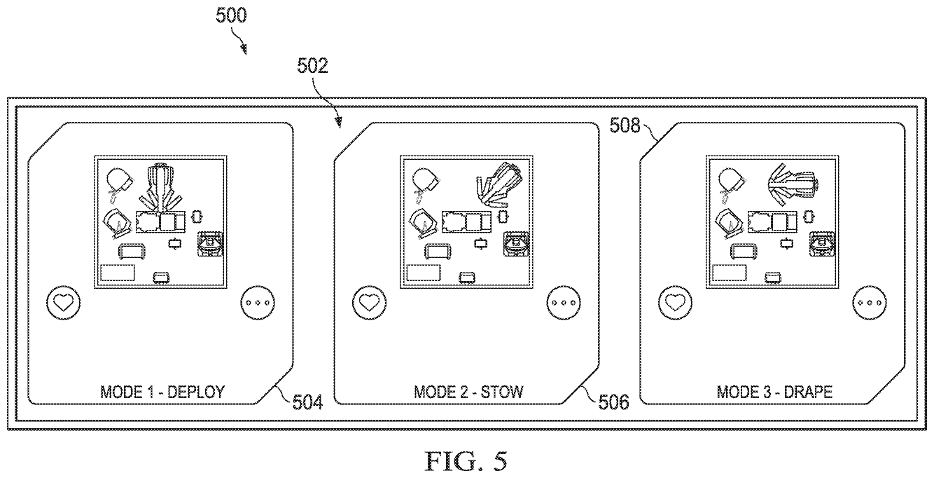

Referring again to FIG. 1, at a process 106, an indicator may be received for a mode of operation of the component to be used in the medical environment. For example, if the component identified for use in the medical environment is the robot-assisted manipulator assembly 404, modes of operation that may be indicated include a sterile preparation mode in which sterile and non-sterile areas of the medical environment are defined. The sterile and non-sterile areas may be any two or three-dimensional areas within the medical environment. In the sterile preparation mode, the manipulator assembly may be arranged to receive a sterile drape, and the draping may be arranged over the manipulator assembly. In some embodiments, the draping procedure may include multiple choreographed steps. The modes of operation may also include a procedure mode in which the draped manipulator assembly is prepared to perform a medical procedure. Other modes of operation may include a trouble-shooting mode in which the mid-procedure manipulator assembly requires attention by an operator to change an instrument or correct a performance issue with the manipulator assembly and a servicing mode in which the manipulator assembly receives routine maintenance or repair service. Other modes of operation may include an inspection mode in which manipulator is inspected for damage and compliance to manufacturer's standards; a cleaning mode in which the manipulator assembly is disinfected, sterilized, or otherwise cleaned; and a storage mode in which the manipulator assembly is stowed before and after a medical procedure or otherwise out of use. The indicator may be generated by a user input received, for example, on a mobile device. In some examples, the user may indicate one or more modes by choosing from a menu. As shown in FIG. 5, a user input device 500, such as a touch screen display, may provide a menu 502 of operational modes for a robot-assisted manipulator assembly 404. As shown, the menu options include a deploy mode 504, a stow mode 506, and a drape mode 508. Each mode may correspond to a configuration of the robot-assisted manipulator assembly including positions and orientations of manipulator arms, set-up joint orientations, and coupled instruments. The mode may also correspond to a collection of components to be positioned within the medical environment to support the mode of operation of the robot assisted manipulator assembly. The mode may also correspond to people including a patient, a surgeon, and/or staff that may be located within the medical environment during the mode of operation of the robot-assisted manipulator assembly.

At a process 108, a set of operation constraints may be received. The operation constraints may be received, for example, from the operation constraint source 418, the component in the medical environment, a user input, and/or other sources with information about the component constraints under the indicated mode of operation. The received operation constraints may include kinematic information for each component, information about the size and shape of the spatial envelope needed for the mode of operation, needed auxiliary components, needed utility access, needed personnel access, needed traffic flow access, patient information, and/or staffing requirements. For example, if the manipulator assembly 404 is the component to be used in the medical environment 200 and the indicated mode is a deploy mode, the received operation constraints may include the initial set-up joint configuration; the manipulator arm range of motion; the types and range of motion of attached instruments; the needed auxiliary components in the medical environment such as an anesthesia cart, display systems, and furniture; utility needs including power, water, air, or Wi-Fi; patient information including weight, age, and medical history; staffing information including number of attendants, type of attendants, and access requirements; environmental conditions including temperature and/or humidity in the medical environment, and traffic information including movement of components and personnel during the deployment of the manipulator assembly.

At a process 110, an environment preparation plan may be generated based on the set of operation constraints, the spatial information, and optionally the guidance information from the guidance source. In some examples, generating the environment preparation plan may include determining which components to include in the medical environment for the mode of operation, determining clearance distances needed between components for the mode of operation, determining a two-dimensional or three-dimensional spatial envelopes for the component range of motion during the mode of operation, determining direction of operator access, determining space needed for operator interaction with the component, determining needed utility access and any peripherals (e.g., cables, connectors) needed for access, determining needed lengths of needed interconnects (e.g. cables, connectors) and preferred amount of interconnect slack; determining traffic patterns; and/or determining personnel workflows and corresponding needed access to entryways, exits, furniture, and components. Generating the environment preparation plan may include, for example, generating a static or animated plan including a two-dimensional and/or three-dimensional model or floor plan for arranging the one or more components and any auxiliary component in the medical environment, identification of sterile and non-sterile areas in the medical environment, sequential instructions for set-up, and traffic and access maps for personnel. The environment preparation plan may include avatars or other markers indicating locations and movement of personnel. Generating the environment preparation plan may include generating warnings unique to the mode or spatial boundaries of the medical environment or may include generating notifications that the mode of operation cannot be safely or effectively performed within the spatial boundaries of the medical environment. In some examples, the warnings may include warnings for non-sterile personnel to avoid sterile areas of the environment and/or may identify sterile areas at risk for breach or procedure sequences that risk breaching sterile areas

Guidance information from the guidance source 419 may be accessed or referenced to prepare the environment preparation plan. The guidance information may include stored information including model medical environment layouts for various modes, manufacturer's instructions for performing the various modes, expert guidance for performing the various modes, best practices related to prior operation of the component in the indicated mode of use, and alerts related to issues identified in prior operation of the component in the indicated mode of use.

In some examples, generating the environment preparation plan includes determining a threshold distance between two components during the mode of operation. The threshold distance may be obtained, for example, from the guidance source. In the environment preparation plan the two components may be arranged so that they are at least a minimum threshold distance apart or no more than a maximum threshold distance apart. If, for example, there is no arrangement of components that satisfies the threshold distance, the generated environment preparation plan may include a warning or may generate an error and an explanation for why the environment preparation plan may not be generated. For example, a threshold distance may be determined between a manipulator assembly and an instrument cart or between a manipulator assembly and a patient table. In some examples, generating the environment preparation plan includes determining an access direction for a component during the mode of operation and comparing the determined access direction to a predetermined access direction (e.g. provided by the guidance source) for the mode of operation.

In some examples, the environment preparation plan may be generated by the control system (e.g., control system 408) or by the guidance source (e.g. guidance source 419). In some examples, the guidance information may be provided by an advisory operator which may be an individual advisor, an advisory network, or an advisory panel to support the generation of the environment preparation plan. The advisory operator may be remote and take the form of tele-connected trainers, mentors, supervisors, or other experts who may, for example, provide instructions, suggestions, options, advisories, and warnings for generating the environment preparation plan. The advisory operator may provide real-time input to the generation of the environment preparation plan or work collaboratively with personnel at the medical environment. Alternatively, the environment preparation plan may be requested and delivered at spaced apart times including hours or days.

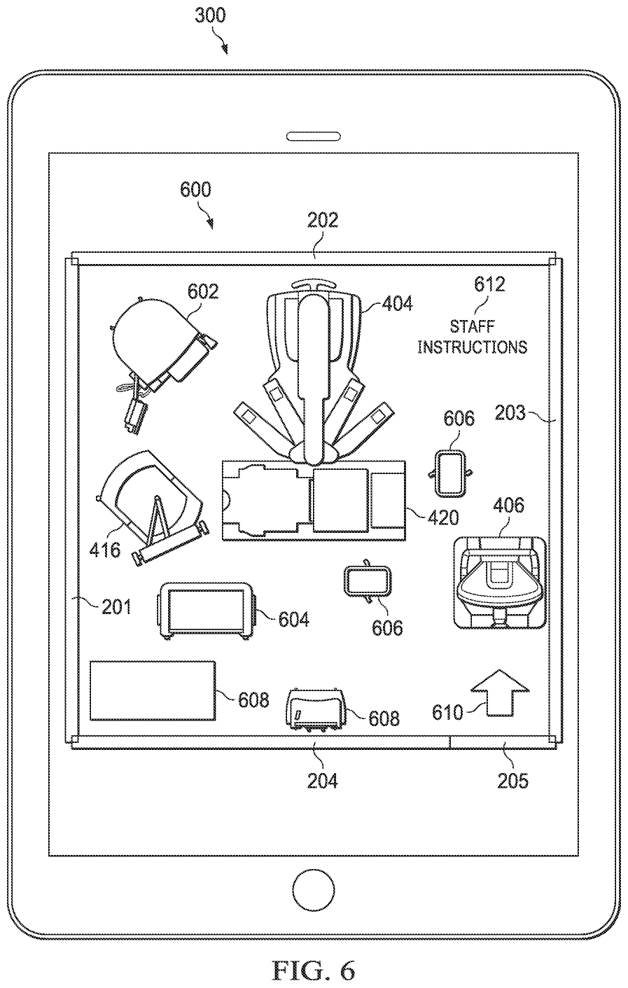

At a process 112, the environment preparation plan may be displayed on a display in the medical environment such as the display system 416. Alternatively or additionally, the environment preparation plan may be displayed on a mobile device in the medical environment or proximate to the medical environment. The mobile device may include an application that displays the environment preparation plan. In some embodiments, the application that displays the environment preparation plan may be the same application that prompted the user to record or capture spatial information. The environment preparation plan may be displayed in a two-dimensional view or in a three-dimensional view. In some embodiments, a user may toggle between two-dimensional and three-dimensional views. FIG. 6 illustrates the user display and input device 300, such as a touch screen display on a mobile phone, that displays an environment preparation plan 600 for a procedure mode of the manipulator assembly 404 within walls 201, 202, 203, 204. Components used in the procedure mode of the manipulator assembly 404 include the operator interface system 406, the display 416, the patient table 420, and auxiliary components including an anesthesia cart 602, an instrument cart 604, tables 506, and furniture 608. The environment preparation plan 500 may illustrate an optimized arrangement of all of the environment components for the spatial boundaries provided by the walls. An optimized arrangement may include, for example, staff access to all component areas that may require access during the procedure mode, components placed proximate to utilities used during the procedure mode, traffic patterns that minimize staff travel times and path intersections, and space to allow a full range of motion for the manipulator and any coupled instruments during the procedure mode. The environment preparation plan may also include renderings (e.g., avatars) of virtual staff members, the surgeon, and/or the patient, including, for example, traffic routes, sterile areas, access paths, personalized instructions or other guidance for personnel placement or movement. The environment preparation plan may include annotations or graphical markers such as symbols, color indicators, animated indicators to provide additional guidance. For example, directional indicators 610 may be used to indicate a travel path or component movement during the mode of operation. Any components or indicators may be animated or rendered in an artificial color to attract a viewer's attention or provide additional information. Annotations may also be included to provide additional information or instruction.

In some embodiments, the environment preparation plan may be displayed to a remote user (e.g., in a different location from the medical environment) who may be a remote mentor, a supervisor, a trainer, an expert advisory group, or other individual or group who may have an interest in the plan and/or a subsequent implementation.

At a process 114, that may be optional, an implementation of the environment preparation plan is evaluated. For example, after components in the medical environment are arranged in preparation for the mode of operation, an evaluation may be performed to determine whether or to what extent the actual arrangement of the components in the medical environment matches the environment preparation plan. The evaluation may be based on kinematic information received from the arranged components, including for example the manipulator assembly, and/or images of the medical environment received after the components are arranged. Additionally or alternatively, the evaluation may be performed by an advisory operator, such as the advisory operator involved at process 110 with the generation of the environment preparation plan. Additionally or alternatively, the evaluation may be performed based on objective or subjective criteria by the surgeon, by a surgical mentor, or by a supervisory panel or individual. For example, after conducting the procedure, the surgeon may save and identify the procedure plan as being associated with a positive patient outcome, an efficient outcome, or any other positive or negative indicator that may be referenced for use in developing subsequent preparation plans.

Additionally or alternatively, the implemented procedure may be evaluated based on performance metrics. Performance metrics may be generated during and/or after the implementation of the procedure. As the plan is implemented, diversions from the generated plan may occur due to expected and unexpected circumstances such as conditions encountered in the patient anatomy, manipulator performance, staff response to encountered conditions with the patient or manipulator, and/or surgeon response to encountered conditions with the patient or manipulator. During the implementation of the procedure, performance metrics may include kinematic information generated about the robot-assisted manipulator assembly and/or the attached instruments and may include structural information such as the dimensions of the components of the manipulator assembly and/or medical instruments, joint arrangement, component position information, component orientation information, and/or port placements. Kinematic information may also include dynamic kinematic information such as the range of motion of joints in the teleoperational assembly, velocity or acceleration information, and/or resistive forces. The structural or dynamic kinematic constraint information may be generated by sensors in the teleoperational assembly that measure, for example, manipulator arm configuration, medical instrument configuration, joint configuration, component displacement, component velocity, and/or component acceleration. Sensors may include position sensors such as electromagnetic (EM) sensors, shape sensors such as fiber optic sensors, and/or actuator position sensors such as resolvers, encoders, and potentiometers.

Performance metrics may also include elapsed times for the overall procedure, elapsed times for each or a plurality of the discrete sub-units of the procedure, elapsed times to complete activities such as a tool change or a maintenance activity, quantity and duration of breaches or near breaches (e.g. based on a breach distance threshold) of a sterile area, and/or tracked movement of personnel as compared to planned movements. In some examples, performance quality metrics may include the accuracy of a human response in complying with instructions. For example, if a tool change is indicated for a first manipulator arm, but a staff member instead changes the tool at a second manipulator arm, the performance metric may indicate a non-compliance with instructions. The performance metrics may be binary metrics such as compliance/non-compliance or may be continuous. Continuous metrics may include, for example, a total staff distance travelled, a quantity of mistakes or unplanned incidents during a procedure, a quantity of instruments used, a quantity of instruments damaged, a quantity of tools changed, or a severity metric to describe the types of human interventions required during the implemented procedure.

Performance metrics may also include post-procedure measures including patient outcome quality metrics such as blood loss during and/or after procedure, patient recuperation time, patient readmissions to a medical facility for a related complication, and patient time to discharge, patient post-procedure infection. Post-procedure measures may also include any damage or measures of wear on the manipulator assembly.

The performance metrics may be compared to standards developed by expert surgeons and staff, benchmarks developed by analyzing multiple prior procedures, and or the standards provided by the planned procedure. For example, the kinematic information from the implemented procedure may be compared to the kinematic information recommended by the procedure plan and an evaluation may be made as to whether and as to what extent the implemented kinematic information matched the planned kinematic information. In some examples, the evaluation may generate a score. Evaluation of the performance metrics, including the elapsed times, may provide an indication of where (e.g., in which sub-unit) in the procedure delays or mistakes occurred. In some embodiments, evaluating the implemented procedure may include comparing the performance metric to benchmark metric and identifying a suboptimal result such as a delay or a mistake. In some embodiments, evaluating the implemented procedure may include comparing the performance metric to benchmark metric and identifying a model result that compares favorably to the benchmark metric.

The results of the evaluation at process 114 (including the initial preparation plan) may be stored for reference or selection in later procedures performed by the same surgeon or different surgeons. The results may also be delivered to a hospital, a medical group, an advisory organization, the system manufacturer, and/or other individuals or groups who may use the evaluation alone or in aggregate for a variety of purposes including to improve the quality of the inputs in the generation of preparation plans. Both suboptimal results and model results from the evaluation of the implemented procedure may provide useful information for improving subsequent procedures.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A system comprising:
a processor; and
a memory having computer readable instructions stored thereon, the computer readable instructions, when executed by the processor, cause the system to:
receive spatial information for of a medical environment;
determine a component for use in the medical environment;
receive an indicator for a mode of operation of the component;
receive a set of operation constraints for the component for the mode of operation; and
generate an environment preparation plan based on the set of operation constraints and the spatial information.

2. The system of claim 1, wherein the spatial information is received from a camera in the medical environment.

3. The system of claim 1, wherein the spatial information is received from a three-dimensional depth mapping system.

4. The system of claim 1, wherein the spatial information is received from a sensor system.

5. The system of claim 1, wherein the determined component is a robot-assisted manipulator assembly.

6. The system of claim 1, wherein the mode of operation is a preparation mode, a procedure mode, a storage mode, or a servicing mode.

7-9. (canceled)

10. The system of claim 1, wherein the set of operation constraints includes a spatial envelope for a range of motion of the component.

11. The system of claim 1, wherein the set of operation constraints includes kinematic information for the component.

12. The system of claim 1, wherein the set of operation constraints includes auxiliary component requirements.

13. The system of claim 1, wherein the set of operation constraints includes utility access requirements for the component.

14. The system of claim 1, wherein the set of operation constraints includes operation access requirements.

15. The system of claim 1, wherein the set of operation constraints includes staffing or patient requirements for the mode of operation.

16. (canceled)

17. The system of claim 1, wherein generating the environment preparation plan includes comparing a distance between the component and a second component to a threshold distance.

18. The system of claim 1, wherein generating the environment preparation plan includes comparing an access direction for the component to a predetermined access direction for the mode of operation.

19. The system of claim 1, wherein generating the environment preparation plan includes determining at least one auxiliary component for use in the medical environment.

20. The system of claim 1, wherein generating the environment preparation plan includes providing a suggested configuration for the component in the medical environment.

21. The system of claim 1, wherein generating the environment preparation plan includes receiving input from a remote advisory operator.

22. The system of claim 1, wherein the computer readable instructions, when executed by the processor, cause the system to display the environment preparation plan on a display system.

23. The system of claim 1, wherein the computer readable instructions, when executed by the processor, cause the system to evaluate an implementation in the medical environment by comparison to the environment preparation plan.

24. The system of claim 1, wherein the computer readable instructions, when executed by the processor, cause the system to store the environment preparation plan for use or reference in a generation of a second environment preparation plan or to store the environment preparation plan with an evaluation indicator based on an implementation.

25. (canceled)
